(19) United States
(12) Patent Application Publication
(10) Pub. No.: US 2005/0226908 A1
(43) Pub. Date: Oct. 13, 2005

(54) Efficacious composition of a benzimidazole, an avermectin and praziquantel and related methods of use

(75) Inventors: Sebastien Claude Jacques Huron, Salisbury, MD (US); Celia B. Shelton, Lewes, DE (US); Joseph Bernard Prullage, Lewes, DE (US)

Correspondence Address: INTERVET U.S.;PATENT DEPARTMENT, PO BOX 318, MILLSBORO, DE, 19966-0318, US

(73) Assignee: Akzo Nobel N.V.

(21) Appl. No.: 11/100,982

(22) Filed: Apr. 6, 2005

Related U.S. Application Data
(60) Provisional application No. 60/620,279, filed on Oct. 19, 2004.
(60) Provisional application No. 60/560,765, filed on Apr. 7, 2004.

(51) Int. Cl. A61K 31/7048; A61K 31/4184

(52) U.S. Cl. 424/442; 514/28; 514/394

(57) ABSTRACT

Embodiments of the present invention generally comprise compositions comprising benzimidazole, avermectin and praziquantel for the control of various parasites and for the prevention of heartworm disease in dogs.

### FIELD OF THE INVENTION

Compositions for the treatment and control of various parasites in animals and related methods of use and manufacture.

### BACKGROUND OF THE INVENTION

Pharmaceuticals have been administered to organisms to control parasites for a number of years. The various pharmaceuticals administered have been effective for different parasites. In response to this, the art field has developed numerous combinations in an effort to treat the varieties of parasites endemic in organisms. These combinations have proved very effective. However, some problems have arisen with interference of the various components (pharmaceuticals). At times, this interference has caused the components to not function as the components would if the components been administered separately. Likewise, and very often unexpectedly, a combination of components can have a synergistic effect on the function. An example of a synergistic effect would be where the combination of components caused one of the components to function better than reported as functioning separately.

Of the numerous combination in the art, the composition of a benzimidazole, an avermectin and praziquantel does not exist in the art. Accordingly, the art field would desire of such a combination.

Benzimidazoles are a well known class of anthelmintic agents which are widely used for the control of endoparasites, particularly nematodes, in domestic animals. Albendazole is indicated for use in humans. Whilst these agents have been used successfully, a number of these anthelmintic benzimidazoles are degraded by oxidative mechanisms in vivo, which accelerates their excretion and this may limit their efficacy and may mean that periodic administrations are necessary.

The avermectin family, of which ivermectin is a member, is a series of very potent antiparasitic agents which are useful against a broad spectrum of endoparasites and ectoparasites in mammals. Ivermectin is disclosed in U.S. Pat. No. 4,199,569, issued Apr. 22, 1980 to Chabala and Fisher. Ivermectin is a mixture, in the ratio of approximately 80:20 of 22,23-dihydroavermectin C-076 B1a and B1b.

Praziquantel is a prazinoisoquinoline derivative anthelmintic used in most schistosome and many cestode infestations (tapeworms). It may be given orally, parenterally or by nasal spray.

A variety of patents exist in the art field concerning parasites and their treatment. Such patents include U.S. Pat. Nos. 6,764,999; 6,753,324; 6,680,308; 6,645,192; 6,596,714; 6,541,037; 6,469,067; 6,426,333; 6,383,471; 6,340,672; 6,193,989; 6,162,820; 5,962,499; 5,925,374; 5,840,324; 5,782,799; 5,776,982; 5,776,981; 5,782,719; 5,637,603; 5,449,681; 5,439,924; 5,340,804; 5,135,953; 5,036,069; 4,963,141; 4,916,120; and, 4,865,598, which are all hereby incorporated by reference.

Various medicaments, including avermectin compounds/milbemycin compounds, have traditionally been administered orally or by injection (subcutaneous, intramuscular) to animals, including humans. In the context of livestock and/or feedstock animals, i.e. meat-producing animals, such avermectin compounds/milbemycin compounds are sometimes added to the animals' food. Such oral administration, however, does not effectively deliver the proper dosage to each and every animal. Significantly, animals that are sick often do not eat or drink properly. These sick animals, however, may be in greatest need such medicaments, including one or more avermectin compounds/milbemycin compounds. Accordingly, the art field is in search of a proper dosage form whereby an effective dose of the various components is administered to the organism.

Chewable dosage forms for drug delivery are well known to pharmaceutical technology. It is known in the pharmaceutical industry that the act of chewing increases the surface area of the available active ingredient and may increase the rate of absorption by the digestive tract. Chewable systems are also advantageous where it is desirable to make an active ingredient available topically to the mouth or throat areas for both local effects and/or systemic absorption. Further, chewable dosage forms are also utilized to ease drug administration in pediatric and geriatric patients. Examples of chewable dosage forms may be found in U.S. Pat. Nos. 6,387,381; 4,284,652; 4,327,076; 4,935,243; 6,270,790; 6,060,078; 4,609,543; and, 5,753,255.

Palatability and “mouth feel” are important characteristics to be considered in providing a dosage form, or matrix, for an active pharmaceutical or medicinal. Unfortunately, many pharmaceuticals and other active ingredients have a bitter or otherwise unpalatable taste, or an unacceptable mouth feel, due to the grittiness or chalkiness of the compound, or both. These characteristics make it difficult to incorporate such active ingredients into the current state of the art for chewable dosage forms because the objectionable taste and/or mouth feel make it less likely to obtain compliance by the user.

As a result, several approaches have been tried in attempting to overcome these problems. The poor taste of a pharmaceutical or other active ingredient may be masked by using suitable flavoring compounds and/or sweeteners. Encapsulation of the active ingredient may also serve to mask bitterness and other undesirable tastes. However, these approaches do not affect the physical state of the dosage form currently employed in the art. For example, chewable vitamin tablets are typically prepared as a compressed, compacted tablet, incorporating one or more active ingredients (e.g., vitamins), a sweetener and flavoring agent to mask the taste of the active ingredients, and a binder, typically microcrystalline cellulose.

Generally, chewable tablets are made by direct compression of a mixture of tableting compounds including the active ingredient, flavorant, binders, etc. The mixture is fed into a die chamber of a tablet press and a tablet is formed by direct compaction. Hardness of the resulting tablet is a direct function of the compression pressure employed. A softer tablet, having an easier bite-through, may be prepared by adding a disintegrant, such as alginic acid, to the pre-tablet mix. Alternatively, a softer tablet may be formed by employing reduced compression pressures. In either case, the resultant tablet is softer, fragile, brittle and easily chipped. Compressed, chewable tablets generally suffer from less than desirable mouth feel, i.e., chalkiness, grittiness, and a dry, powdery taste. Antacid tablets, e.g., Tums.RTM. manufactured by SmithKline Beecham Corp., Pittsburgh, Pa. and Rolaids.RTM. manufactured by Warner Lambert of Morris Plains, N.J., are each examples of typical compressed chewable tablets.

Attempts have been made to reduce the grittiness and/or chalkiness of the compressed tablet by coating particles of the active ingredient with oils or fats, which coat the particles prior to incorporation into the delivery system. In this way, the grittiness or chalkiness of the particles is masked by the oil or fat while the particles are in the mouth. In addition, tablet softness is improved. After swallowing, the oil or fat is removed and the particle can be absorbed by the digestive system. However, the addition of fats or oils to the pre-tablet mix can cause the tableting ingredients to adhere to the die chamber and cause a reduction in the binding action of the binders present in the mix. Accordingly, the art field is in search of a process of manufacturing a soft chew whereby compression and subsequent product loss may be minimized or lessened.

Other techniques for providing a chewable delivery system involve the use of a gum base. Gum bases are insoluble elastomers which form the essential element for chewing gum. The gum base is typically blended with one or more sweeteners to obtain a confectionery gum. A coating containing the active ingredient is then applied over the confectionery gum. As the dosage form is chewed, the coating fractures and/or is dissolved in the mouth and swallowed.

Other delivery systems involve the use of layered, non-homogeneous structures.

Another chewable delivery system is based on a nougat-type, chewy tablet. Such tablets generally employ a base of corn syrup (or a derivative). Such tablets are prepared as a confectionery, i.e., the corn syrup is cooked with water and a binder such as soy protein.

However, the art field has experienced problems with delivering additives/active ingredients to organisms because of palatability issues. Complex guidelines exist along the regulatory framework that make it very difficult to make and/or manufacture a palatable composition with an additive. Accordingly, the art field is in search of a method and/or composition of delivering an additive to an organism in a palatable format.

One part solution is in U.S. Pat. No. 6,387,381 (hereinafter referred to as the '381 patent). The '381 patent discloses an extrudate with formed of a matrix having starch, sugar, fat, polyhydric alcohol and water in suitable ratios such that there exists a water activity of 0.6-0.75, for carrying an active ingredient. The water activity of the product matrix may be adjusted up or down for the active ingredient, be it pharmaceutical, nutraceutical, or a vitamin mineral complex. The claimed product is directed towards a product containing an additive, an extrudate comprising a matrix having about 10 to about 50% wt starch, a sweetener consisting essentially of sucrose, corn syrup and sorbitol, said sucrose being in an amount of at least 10%, and at least about 5% wt water, said composition having A_{w} of about 0.60 to about 0.75, and a soft and chewy texture, and said A_{w} being adjusted to permit an appropriate amount of free water in the presence of the additive. However, this product is limited to an extrudate and not available in a tablet form of formulation.

### SUMMARY

In general, embodiments of the present invention relate to a palatable composition comprising a benzimadizole, an avermectin and praziquantel combination. A variety of dosage forms are disclosed herein, most preferred being a soft chew dosage form. Various embodiments of the present invention are useful for the periodic treatment of parasites in organisms.

The combination of the composition produces a synergistic effect in the treatment of organisms based on previous publications that concluded that one time treatment with fenbendazole at 100 or 150 mg/kg was not efficacious. See JAVMA 180, 1:53 (January 1982) and Am J Vet Res 39, 11:1799 (November 1978).

Also disclosed are processes of manufacture for the composition.

### BRIEF DESCRIPTION OF THE DRAWINGS

FIG. 1a is an illustration of an embodiment of a composition of the present invention.

FIG. 1b is an illustration of an alternate embodiment of a composition of the present invention.

FIG. 2 is an illustration of an embodiment of a forming apparatus used in forming various embodiments of compositions of the present invention.

FIG. 3 is an illustration of an embodiment of knock out used to form an embodiment of the present invention.

### DETAILED DESCRIPTION OF THE INVENTION

As used herein, the term “cookie” and “soft chew” and any conjugation thereof, means and refers to an edible composition.

As used herein, the term “sugar,” and any conjugation thereof, means and refers to any saccharide which is at least partially soluble in moisture, non-toxic, and preferably not provide any undesirable taste effects. Further, the use of the term “sugar” shall include a “sugar substitute.”

As used herein, the term “sugar substitute,” and any conjugation thereof, means and refers to any compound that produces a like effect as sugar, but does not require the same or magnitude of effect that a comparable amount of a sugar would produce.

As used herein, the term “parasite,” and any conjugation thereof, means and refers to species of organism treated by a pharmaceutical, non-limiting examples of which are included herein. Internal and external parasites of Equidae, Canidae, Felidae, Bovidae, Ovidae, Capridae, Suidae which include but are not limited to the pseudophyllidean and cyclophyllidean tapeworms, digenean flukes nematodes in the orders Rhabditida, Strongylida, Oxyurida, Ascaridida, Spirurida and Enoplida.

Exemplary, non-limiting parasites of equine include, but are not limited to, large strongyles, such as, but not limited to Strongylus vulgaris, S. edentatus, and S. equines; small strongyles including, but not limited to, those resistant to some benzimidazole class compounds, Triodontophorus spp., Cyathostomum spp., Cylicocyclus spp., Cylicostephanus spp., and Cylicodontophorus spp.; pinworms, such as, but not limited to Oxyuris equi; Ascarids, such as, but not limited to, Parascaris equorum; Hairworms, such as, but not limited to, Trichostrongylus axei; largemouth stomach worms, such as, but not limited to Habronema muscae; neck threadworms, such as, but not limited to Onchocerca spp.; bots, such as, but not limited to, Gastrophilus spp.; lungworms, such as, but not limited to, Dictyocaulus arnfieldi; intestinal threadworms, such as, but not limited to, Strongyloides westeri; summer sores caused by Habronema and Draschia spp., and other cutaneous larvae; and other parasites that are common in the art. However, parasites of other species are specifically contemplated as falling within the scope of the invention.

Exemplary, non-limiting parasites of canines comprise Toxocara canis, Toxascaris leonina, richuris vulpis, Ancylostoma caninum, Ancylostoma braziliense, Uncinaria stenocephala, Dirofilaria immitis, Angiostrongylus vasorum, Filaroides osleri, Filaroides hirthi, Filaroides milksi, Physaloptera spp., Eucoleus aerophilus, Pearsonema plica, Dracunculus insignis, Nanophyetus salmincola, Paragonimus kellicotti, Dipylidium caninum, Taenia pisiformis, Taenia hydatigena, Taenia multiceps Taenia ovis, Echinococcus granulosus, Echinococcus multilocularis, Mesocestoides spp., Diphyllobothrium latum, and Giardia spp.

Exemplary, non-limiting parasites of felines comprise Toxocara cati, Toxascaris leonine, Ancylostoma tubaeforme, Uncinaria stenocephala, Dirofilaria immitis, Angiostrongylus vasorum, Aelurostrongylus abstrusus, Eucoleus aerophilus, Pearsonema plica, Aonchotheca putorii, Dracunculus insignis, Paragonimus kellicotti, Dipylidium caninum, Taenia taeniaeformis, Spirometra spp., Echinococcus granulosus, Echinococcus multilocularis, Mesocestoides spp., Diphyllobothrium latum, and Giardia spp.

These lists were only to serve as examples. The species of animals listed and the parasites are not limiting. Other animal species and other parasites are contemplated, as would be understood by one of ordinary skill in the art.

As used herein, the term “amylaceous ingredients” is meant those food-stuffs containing a preponderance of starch and/or starch-like material. Examples of amylaceous ingredients are cereal grains and meals or flours obtained upon grinding cereal grains such as corn, oats, wheat, milo, barley, rice, and the various milling by-products of these cereal grains such as wheat feed flour, wheat middlings, mixed feed, wheat shorts, wheat red dog, oat groats, hominy feed, and other such material. Also included as sources of amylaceous ingredients are the tuberous food stuffs such as potatoes, tapioca, and the like.

As used herein, percents of components of the soft chew means and refers to percentages of the total weight of the soft chew.

As used herein, the term “starch component” shall mean and refer to a starch or starches component and is considered a dry component, whether or not actually dry. As used herein, the term “sugar component” shall mean and refer to a sugar or sugars and/or sugar substitute component and is considered a dry component, whether or not actually dry. As used herein, the term “oil component” shall mean and refer to an oil or oils component and is considered a liquid component, whether or not actually liquid. As used herein, the term “additive component” shall mean and refer to an additive or additives. As used herein, the term “emulsifier component” means and refers to an emulsifier or emulsifiers, humectants and the like and is considered a liquid component, whether or not actually liquid.

Embodiments of the present invention generally comprise a composition comprising the components of a benzimadizole, an avermectin and praziquantel to an organism and related methods of use and manufacture. In an embodiment, the composition comprises about 0 mg to about 1000 mg fenbendazole per kilogram (kg) body weight of the organism, about 0 mcg to about 0.5 g ivermectin per kilogram (kg) body weight of the organism, and about 0 mcg to about 0.5 g praziquantel per kilogram (kg) body weight of the organism. In an alternate embodiment, the composition comprises about 25 mg to about 500 mg fenbendazole per kilogram (kg) body weight of the organism, about 2 mcg to about 0.2 g ivermectin per kilogram (kg) body weight of the organism, and about 2 mcg to about 0.2 g praziquantel per kilogram (kg) body weight of the organism. In an alternate embodiment, the composition comprises about 50 mg to about 300 mg fenbendazole per kilogram (kg) body weight of the organism, about 3 mcg to about 0.1 g ivermectin per kilogram (kg) body weight of the organism, and about 3 mcg to about 0.1 g praziquantel per kilogram (kg) body weight of the organism. In an alternate embodiment, the composition comprises about 100 mg fenbendazole per kilogram (kg) body weight of the organism, about 6 mcg ivermectin per kilogram (kg) body weight of the organism, and about 5 mg praziquantel per kilogram (kg) body weight of the organism.

The components may be delivered in any suitable pharmaceutical carrier. Various embodiments may be delivered as a liquid, a solid, a vapor, a gel, and/or any other pharmaceutical delivery form. Quite often the various components are incorporated with a flavoring to make the components more palatable.

Various other embodiments of a composition of the present invention are an edible delivery vehicle or soft chew for the delivery of a composition comprising a benzimadizole, an avermectin and praziquantel to an organism. Especially considered organisms include livestock, pets, farm animals, and the like, including, but not limited to, horses, cows, pigs, goats, sheep, llamas, deer, ducks, chickens, dogs, cats, lions, tigers, bears, oxen, buffalo, fish, birds, and the like. Embodiments of the present invention generally comprise compositions for the control of various parasites including Toxocara canis, Ancylostoma caninum, Trichuris vulpis, Dipylidium caninum and for the prevention of heartworm disease caused by Dirofilaria immitis in dogs, among others.

In various embodiments, the benzimadizole is selected from the group consisting of mebendazole, oxibendazole, fenbendazole, oxfendazole, triclabendazole, flubendazole, ricobendazole, thiabendazole, levamisole and albendazole and its prodrugs. However, other benzimadizoles would be readily apparent to those of ordinary skill in the art, whether presently existing or after arising. Such other benzimadizoles are intended to be covered be the scope of the claims appended hereto.

In various embodiments, the avermectin is any of a group of macrocyclic lactones that are endectocides that are anthelmintics and insecticides. Examples of macrocyclic lactones are abamectin, ivermectin, eprinomectin, selamectin, or milbemycin oxime. In an embodiment, the avermectin is ivermectin.

In a variety of embodiments, the composition is administered in a dosage form, such as a tablet, pill, powder, liquid, paste, soft chew, and the like. Preferred dosage forms are a palatable soft chew. However, other embodiments of the present invention may be used as or in combination with a feed, feed additive, supplement, neutraceutical, pharmaceutical, treat, and the like.

Pending PCT application US03/25358, the contents of which are hereby incorporated by reference, fully discloses and claims a preferred carrier and methods for its manufacture for the claimed compositions.

In an embodiment, a composition of the present invention comprises a starch component, a sugar component, and an oil component. Generally, in various embodiments, the starch component comprises about 5 percent to about 60 percent of the soft chew, the sugar component comprises about 5 percent to about 75 percent of the soft chew, and the oil component comprises about 1 percent to about 40 percent of the soft chew. The percentages of the starch component, sugar component, and/or oil component may be varied depending upon the end use and desired consistency of the soft chew.

In an alternate embodiment, the starch component comprises about 15 percent to about 40 percent of the soft chew, the sugar component comprises about 15 percent to about 60 percent of the soft chew, and the oil component comprises about 5 percent to about 30 percent of the soft chew.

In an alternate embodiment, the starch component comprises about 25 percent to about 35 percent of the soft chew, the sugar component comprises about 25 percent to about 50 percent of the soft chew, and the oil component comprises about 7 percent to about 15 percent of the soft chew.

The starch component may comprise starch from any source and may act as a binder in the soft chew. In an embodiment, the starch component is derivatized and/or pregelatinized. In a preferred embodiment, the starch component is highly derivatized. Some starches that can serve as a base starch for derivatization include regular corn, waxy corn, potato, tapioca, rice, etc. Suitable types of derivatizing agents for the starch include, but are not limited to, ethylene oxide, propylene oxide, acetic anhydride, and succinic anhydride, and other food approved esters or ethers, introducing such chemicals alone or in combination with one another.

In various embodiments, prior cross-linking of the starch in the starch component may or may not be necessary, based on the pH of the system and the temperature used to form the product.

The starch component may also include amylaceous ingredients. The amylaceous ingredients can be gelatinized or cooked before or during the forming step to achieve the desired matrix characteristics. If gelatinized starch is used, it may be possible to prepare the product of the subject invention or perform the process of the subject invention without heating or cooking. However, ungelatinized (ungelled) or uncooked starch may also be used.

The sugar component may act as a sweetener and may comprise sugars including, but not limited to, white sugar, corn syrup, sorbitol (solution), maltitol (syrup), oligosaccharide, isomaltooligosaccharide, fructose, lactose, glucose, lycasin, xylitol, lactitol, erythritol, mannitol, isomaltose, polydextrose, raffinose, dextrin, galactose, sucrose, invert sugar, honey, molasses, polyhydric alcohols and other similar saccharides oligomers and polymers and mixture thereof. In addition, artificial sweeteners such as saccharine, aspartame and other dipeptide sweeteners may be present and sugarless can include solid polyols such as Sorbitol, Mannitol and Xylitol. Examples of various well established sources of a portion of these sugars are, corn syrup solids, malt syrup, hydrolyzed corn starch, hydrol (syrup from glucose manufacturing operations), raw and refined cane and beet sugars, and the like.

The oil component may act as a humectant and may comprise more than one oil including, but not limited to, fat or fats, both natural and synthetic. Oil employed as an ingredient in the soft chew may be a saturated or unsaturated liquid fatty acid, its glyceride derivatives or fatty acid derivatives of plant or animal origin or a mixture thereof. A source for typical animal fats or oils are fish oil, chicken fat, tallow, choice white grease, prime steam lard and mixtures thereof. However, other animal fats are also suitable for use in the soft chew. Suitable sources for vegetable fats or oils can be derived palm oil, palm hydrogenated oil, corn germ hydrogenated oil, castor hydrogenated oil, cotton-seed oil, soybean oil, olive oil, peanut oil, palm olein oil, Cacao fat, margarine, butter, shortening and palm stearin oil, and mixtures thereof. Additionally, a mixture of animal or vegetable oils or fats is suitable for use in the matrix.

Various other embodiments further comprise a flavoring component. Such flavoring component, in an embodiment, is to improve and/or change the palatability of the soft chew. Any flavoring in the flavoring component may be used. Examples of suitable flavor for the flavoring component includes, but is not limited to, strawberry flavor, tutti fruity flavor, orange flavor, banana flavor, mint flavor, and an apple-molasses. A suitable source for an apple-molasses flavoring component is Pharma Chemie, 1877 Midland Street, P.O. Box 326, Syracuse, Nebr. 68446-0326, under a product name of Sweet-Apple Molasses Flavoring, Product Code PC-0555.

In various embodiments, other flavorings for the flavoring component may be used, such as fruit, meat (including, but not limited to pork, beef, chicken, fish, poultry, and the like), vegetable, cheese, cheese-bacon and/or artificial flavorings. In preferred embodiments utilizing a flavoring component, the flavoring component is chosen to enhance the palatability of the composition. A preferred meat flavoring is commercially available at Pharma Chemie as Artificial beef flavor product code PC-0125. A flavoring component is typically chosen based upon consideration related to the organism that will be ingesting the soft chew. For example, a horse may prefer an apple flavoring component, while a dog may prefer a meat flavoring component.

Various embodiments further comprise a stabilizer and/or lubricating component. In an embodiment, suitable stabilizer components are Magnesium Stearate, citric acid, sodium citrate, and/or the like. However, stabilizer components are common in the art and any suitable one or mixture of more than one may be used. In an embodiment, a stabilizer component comprises about 0.0 percent to about 3.0 percent of the soft chew. In an alternate embodiment, a stabilizer component comprises about 0.5 percent to about 1.5 percent of the soft chew.

Various embodiments further comprise an emulsifier component. A suitable emulsifier component is a glycerin, glycerin fatty acid ester, sorbitan monostearate, sucrose fatty acid ester, lecithin, polyethylene glycol, mixtures thereof, and the like. However, emulsifier components are well-known in the art field and any emulsifier component may be used. Generally, the amount of emulsifier component added may affect the stickiness of the soft chew. The greater the concentration of glycerin, the stickier the soft chew. In an embodiment, an emulsifier component comprises about 0.0 percent to about 40 percent of the soft chew. In an alternate embodiment, an emulsifier component comprises about 5.0 percent to about 30 percent of the soft chew. In an alternate embodiment, an emulsifier component comprises about 10 percent to about 20 percent of the soft chew.

In various embodiments, a moisture component is in the composition. In an embodiment, a moisture component comprises about 0.0 percent to about 15 percent. In an alternate embodiment, a moisture component comprises about 2.0 percent to about 10 percent. In an alternate embodiment, a moisture component comprises about 5.0 percent to about 7.5 percent.

The previously disclosed amounts and/or concentrations of the benzimadazole, the avermectin and praziquantel may also be used in a soft chew formulation of the present invention.

In various embodiments, an additive component is added to the composition. The additive component is selected from the group consisting of a pharmaceutical, a nutraceutical, a vitamin, a mineral and/or a filler that can be orally administered. In this regard, an additive component may be an active ingredient or an inactive ingredient.

Various embodiments of the present invention contemplate the composition with additional pharmaceuticals/additives. Exemplary embodiments with more than one pharmaceutical include, without limitation, with an antiparasitic compound with effect on ectoparasites, e.g. aryl-pyrazole compounds, nitenpyram, lufenuron, omeprazole, pyrantel pamoate, selamectin or milbemycin Generally, any drug that is given as a tablet could be put in the composition, as long as the excipients in the formulation wouldn't cause a stability problem or combine with them is such a way as to make them inactive.

Other pharmaceuticals comprise, but are not limited to, ivermectin, fenbendazole, piperazine, magnesium hydroxide, stranozole, furosemide, penicillin, amoxicillin, prednisolone, methylprednisolone, acepromazine, aspirin, PROZAC, ZANTACS, BENADRYL, praziquantel, pyrantel, HOE 12073, Sumitomo Chemicals-1638, Nitenpyram, spinosad and omyprazole.

In various embodiments, the composition is coated. Any suitable coating may be used. In an embodiment, a coating is chosen that will not interfere with an additive. In another embodiment, an additive is chosen that can modify the time for digestion of the additive(s), thereby at least partially controlling the release of the additive(s). Suitable coatings include, but are not limited to, and may be any pharmaceutically acceptable, and/or neutraceutically acceptable coating, as is common in the art. (polymers, monomers). Reference can be had to U.S. Pat. No. 6,498,153, to Cady et al. for a list of polymers that can function as coatings.

Exemplary nutraceuticals, vitamins, minerals, and the like include, but are not limited to, vitamins such as vitamin A, vitamin B₁, vitamin B₂, vitamin B₆, vitamin B₁₂, vitamin C, vitamin D, vitamin E, vitamin K, nicotinamide, folic acid, calcium pantothenate, biotin and mixtures thereof; mineral supplements such as calcium, calcium carbonate, calcium phosphate, magnesium, magnesium carbonate, magnesium glycerophosphate, manganese, potassium, lecithin, iron, copper, zinc, phosphorus, hippophae rhamnoides ext., pollen, Garcinia, Echinaceae, ginsenoside ext., Ginkgo biloba ext., blueberry, hawthorn ext., acanthopanax ext., aloe ext., Cardus marianus ext., chromium picolinate, potassium gluconate and methionine amino acid, iron, copper, zinc, and mixtures thereof.

Exemplary fillers include, but are not limited to, a carbohydrate source, a protein source, antioxidants, such as Tenox 8, gum, colorants, dyes, pigments, and the like. Generally, any ingredient may be used as a filler. In preferred embodiments, the filler is chosen so as not to adversely affect the palatability of the soft chew.

An embodiment of a process for forming a soft chew of the present invention comprises the steps of:
mixing a starch component, a sugar component, an oil component, and the composition;
optionally heating at least a portion of the components; and,
forming embodiments of the soft chew.

In further embodiments, additional additives are mixed.

The composition, and additional additives (if present) may be mixed along with the other components or at a later step and/or time in the process. In an alternate embodiment, the components are mixed completely to produce a mixed dough. In a most preferred embodiment, the dough is mixed until there is a uniform dispersal of the components in the dough.

In a further embodiment, the process further comprises mixing an emulsifier component. The emulsifier component may be chosen to act as a humectant and/or a forming agent. In an embodiment, a forming agent of choice is polyethylene glycol (PEG). Moreover, depending upon the desired consistency of the soft chew, different molecular weight PEG may be utilized. In an embodiment, PEG 3350 is utilized. However, the PEG chosen is a matter of choice and the molecular weight may be higher or lower than 3350.

Embodiments of processes of the present invention may further comprise mixing a stabilizer component, a flavoring component, and/or a filler component.

In an embodiment, the dry components are mixed and the liquid components are mixed separately. In an embodiment, the oil component and the emulsifier component are heated when mixing and added, at sufficient temperature, to the dry components. The liquid and dry components are then mixed together until a desired dough is obtained. However, the process by which the components are mixed and/or heated into a dough may be varied. Moreover, the degree of mixing may be varied, such that, in various embodiments, the dough is not uniformly mixed and remains striated. Likewise, various embodiments of dough of the present invention have discrete zones and/or layers.

In an embodiment, an additive(s) component is added during mixing of the components. In an alternate embodiment, an additive component is injected into the soft chew after forming. In an alternate embodiment, a dough is formed about an additive component. In another embodiment, an additive(s) is mixed and/or dissolved in an alcohol or other liquid prior to adding with a dough and/or components of the present invention. In alternate embodiments, an additive(s) component is sprayed into a dough while mixing. The particular process for mixing the additive in the dough may be dependant upon considerations, including the stability of the additive, the temperature sensitivity of the additive, and/or the like. In an embodiment, the various components and composition are uniformly mixed and/or dispersed in the dough.

In another embodiment, the oil component is heated prior to mixing the components, whereby

The dough is then formed into a soft chew of the present invention by a knockout. In an embodiment, the dough is formed while still warm. The dough may be formed into a soft chew by any means or method common in the art, such as by hand or by machine. In an embodiment, a forming machine or patty machine is utilized, such that the soft chew is formed out of the dough. Suitable examples of forming machines are exemplified in U.S. Pat. Nos. 5,165,218, 7,780,931, 4,523,520, and 3,887,964. A most preferred forming machine is the FORMAX machine manufactured by FORMAX Food Machines, Mokena, Ill.

By knockout is meant that embodiments of soft chews of the present invention are formed from the dough. In an embodiment, the knockouts may be cut from the dough, such as by a cookie cutter knockout. In other embodiments, the knockouts are pressed out of the dough. In yet other embodiments, the knockouts are ripped from the dough. The formation of knockouts will be readily apparent to those of ordinary skill in the art from the disclosure of the present application and the material incorporated by reference.

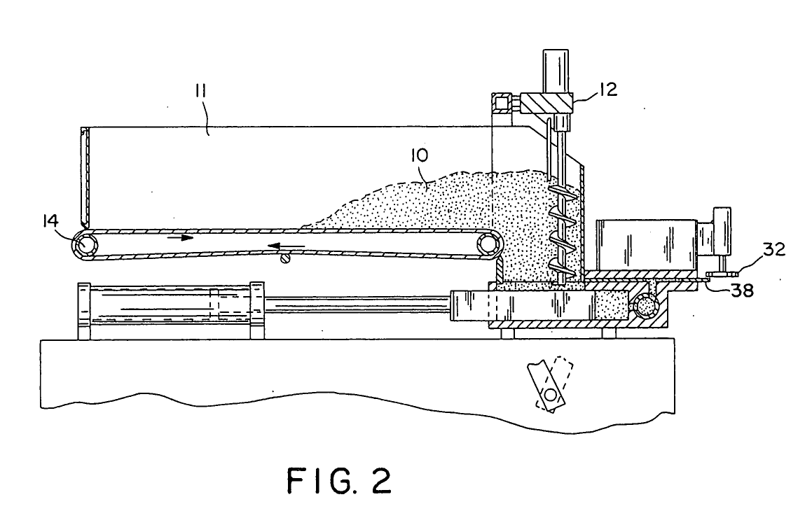

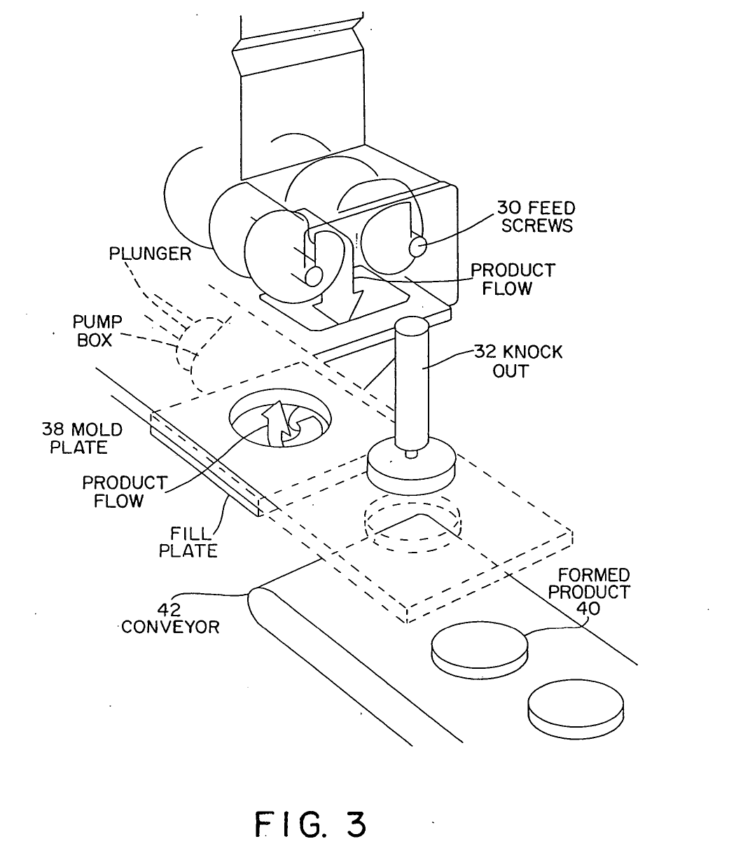

Now referring to FIG. 2, an illustration of an embodiment of a forming apparatus used in forming embodiments of compositions of the present invention, a general preferred embodiment for forming soft chews of the present invention will be discussed. Generally, dough 10 is added to hopper 11. Screw(s) 12 and conveyor 14 move dough 10 through feed screw(s) 30 and onto mold plate 38. Knock out 32 then forms a soft chew of the present invention. Reference to FIG. 3, an illustration of an embodiment of knock out used to form an embodiment of the present invention, illustrates a preferred embodiment of soft chew 40. However, any size or shape knock out is acceptable. Soft chew 40 is then conveyed along conveyor 42. Soft chew 40 may be used, packaged, or as is desired.

In an embodiment, dough 10 is formed into soft chew 40 while still warm. However, dough 10 may be formed into soft chew 40 at any desired temperature.

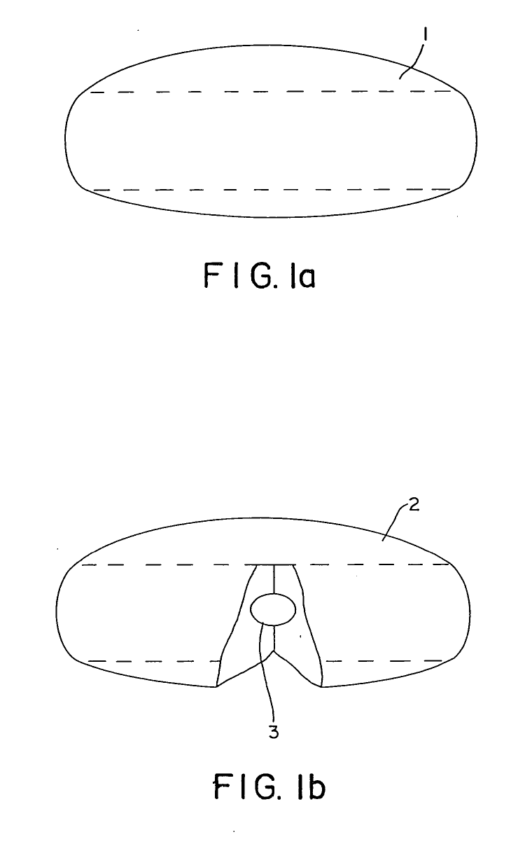

Embodiments of a soft chew of the present invention may have different textures, crispyness, hardness, and the like. In an embodiment texture of the soft chew will be smooth. In other embodiments, the texture of the soft chew will be rough. Now referring to FIG. 1a, an embodiment of a soft chew of the present invention, a soft chew 1 that was formed from a uniformly mixed dough is illustrated. However, FIG. 1b illustrates a soft chew 2 with more than one zone. Whether the dough is uniformly mixed or not may be dependent upon various factors, including the type of additive, consistency of soft chew, and/or the like.

Further embodiments of the present invention are for a process/method of controlling parasites in an organism comprising the steps of administering the composition described above to the organism, the composition comprising a an benzimadizole, an avermectin and praziquantel. The dosage requirements for embodiments of the present invention will vary and should be chosen to be within established veterinary parameters. To control dosage, the composition may be administered at various times. In an embodiment, the composition is administered monthly. In an alternate embodiment, the composition is administered semi-annually. In yet and alternate embodiment, the composition is administered annually. However, the frequency of administration may be varied according to the composition formulated. Generally, the composition may be formulated for any frequency of administration, as is common in the art.

Another manner of controlling dosage is controlling the number of compositions given to an organism. Each composition could be for treating a certain number of pounds of an organism, for example 25 kg. By way of example, and not limitation, a 25 kg organism could receive one dose whereas a 75 kg organism would receive three doses, i.e., dosage delivered to an organism may be adjusted by offering more or less units of soft chews to the organism, one soft chew supplies enough additive for a certain weight organism.

Suitable examples of organisms are livestock, pets, farm animals, and the like, including, but not limited to, horses, cows, pigs, goats, sheep, llamas, deer, ducks, chickens, dogs, cats, lions, tigers, bears, oxen, buffalo, fish, birds, and the like. In an embodiment, the organism is a canine. Particular parasites treated in canine with the above formulation would include, but not limited to, Toxocara canis, Ancylostoma caninum, Trichuris vulpis, Dipylidium caninum and for the prevention of heartworm disease caused by Dirofilaria immitis in dogs. This is surprising, as the art field has indicated that the claimed dosage of the benzimadazole (fenbendazole) component would not be effective against these parasites. The synergistic effect of the fendendazole, ivermectin and praziquantel combination is novel and defies the reported literature. Insert previous mentioned reference?

The following examples illustrate the unexpected results obtained by embodiments of the present invention.

### EXAMPLES

The objective of the studies was to assess the efficacy of a palatable fenbendazole, ivermectin, and praziquantel combination. A soft chew product formulation was chosen and administered at a single dose of 100 mg fenbendazole, 6 mcg ivermectin and 5 mg praziquantel per kg body weight against induced parasitic infections. The studies were conducted in accordance with the FDA Center for Veterinary Medicine (CVM) Guidance Document #85 entitled “Good Clinical Practice, VICH GL9, Final Guidance” (May 9, 2001). Persons involved with the study were provided a copy of the Guidance prior to study initiation.

### Example 1

One study was done to assess the efficacy of a palatable fenbendazole, ivermectin and praziquantel combination soft chew product administered at a single dose of 100 mg fenbendazole, 6 mcg ivermectin and 5 mg praziquantel per kg body weight against induced infections of Ancylostoma caninum. The study was conducted in accordance with the FDA Center for Veterinary Medicine (CVM) Guidance Document #85 entitled “Good Clinical Practice, VICH GL9, Final Guidance” (May 9, 2001). Twenty purpose-bred beagles (10 males and 10 females) were given oral inoculations of approximately 300 A. caninum larvae on Study Day-28 and infections were confirmed by three separate fecal egg counts by Study Day-2. The dogs were ranked in descending order according to their mean fecal egg count and then randomly assigned to one of two groups: Group 1 received a single treatment of the soft chews containing the active veterinary product and Group 2 received a single treatment of the placebo control soft chew product. All of the dogs were administered treatment on Study Day 0. On Study Day 7, all dogs were euthanized and the intestinal contents were collected and the number of hookworms present was counted. The reduction in A. caninum worm recoveries based on geometric means relative to the controls was 98.4%. No treatment-related adverse events were observed. This study demonstrated that a single treatment of a palatable fenbendazole, ivermectin and praziquantel combination soft chew product administered at a single dose of 100 mg fenbendazole, 6 mcg ivermectin and 5 mg praziquantel per kg body weight was efficacious against induced A. caninum infections.

Materials and Methods

A split plot study design was used for this placebo controlled efficacy study. The blocking factor was A. caninum fecal egg counts. Blocks consisted of two dogs, one male and one female, and the dogs were ranked in descending order by mean fecal egg count.

Treatments

The active ingredients in the embodiment delivered to the dogs were fenbendazole (100 mg/kg), ivermectin (6 mcg/kg) and praziquantel (5 mg/kg). The batch number of this experimental formulation was PC005.094. The complete formulation is on file with the Sponsor. The veterinary product was stored in a dry place at controlled room temperature and was protected from light.

Control Product

The control product did not contain any active ingredients. The control product was stored in a dry place at controlled room temperature.

Dosage and Administration

The investigational and control products were in a chewable solid dosage form and each soft chew weighed approximately 5.2 grams to 5.4 grams. The veterinary product contained the active ingredients in sufficient quantity to treat up to 24.5 pounds of body weight. The dogs' weights ranged from 16.3 to 30.3 pounds. Dogs received a single oral treatment of either the veterinary product or the control product. Food was removed from the dogs the night before dosing, and then food was offered approximately 30 minutes prior to dosing. Of those dogs assigned to the veterinary product, two dogs were each administered two chews and the remaining eight dogs were each administered one chew.

Study Procedures

| Study Day | Activity |
|---|---|
| −35 | Placed dogs in individual kennels or cages and start |
| | of acclimation |
| Prior to −28 | Fecal samples collected to confirm dogs were free |
| | of parasites |
| −28 | Dogs inoculated with larvae of A. caninum |
| By −7 | Physical exams; blood samples for hematology and |
| | serum chemistry |
| −5 to −3 | Three fecal samples collected and parasite egg |
| | counts performed. |
| −1 | Weighed dogs for study and assigned dogs to |
| | treatment group |
| 0 | Administered treatment; dogs were observed hourly |
| | for 4 hours post treatment |
| 1-7 | Daily observations |
| 5 | Blood samples for hematology and serum chemistry |
| 7 | Necropsies, parasite collections, and parasite counts |
| | performed. |

Randomization/Allocation to Treatment Group

Enumeration of A. caninum eggs in fecal samples was done for each dog on three separate days by Study Day-2. Dogs were then ranked by their mean fecal egg counts within sex in descending order and randomly assigned to a treatment group (five males and five females per group).

Blinding

The personnel that performed the daily observations, necropsies, and counted parasites recovered at necropsy were blinded to the treatment the dogs received. The individual responsible for administering treatment was not blinded.

Study Variables and Observations

Pre-treatment physical examinations and blood samples were collected by Study Day-7 to establish that dogs were healthy, except for parasitism, prior to inclusion in the study. Blood samples were also collected prior to euthanasia to compare hematology and serum chemistry parameters to the initial values. The blood samples were sent to a clinical pathology laboratory for a complete blood count and serum chemistry profile.

Dogs were inoculated with approximately 300 viable L3 larvae 28 days prior to Study Day 0 as described in Appendix I. The source data for the larvae-donor dog is included in the study data.

All dogs were observed once per hour for adverse events for 4 hours after administration of the veterinary and control product. After this, the dogs were observed once daily until they were euthanized (Study Day 7).

The primary efficacy variable was the number of A. caninum worms recovered at necropsy. Counts of A. caninum were determined as described in the Appendix II. The counts were documented and are included in the study data. There were no secondary variables.

Analytical Methods

Standard laboratory equipment was used in the collection of samples and for the identification of parasites.

Statistical Methods

Parasite counts were transformed by taking the natural logarithm of the count of intestinal parasites recovered from each animal +1. One was added to the count to prevent an undefined result for those dogs with no parasites. The transformed counts were analyzed using the Proc Mixed procedure in the SAS® statistical software by modeling the counts with a mixed model. Treatment and sex were modeled as fixed effects and block was modeled as the random effect. The model is as follows:
Log (Yᵢⱼₖ+1)=μ+bₖ+αᵢ+βⱼ+αβᵢⱼ+αbᵢₖ+βbⱼₖ+eᵢⱼₖ
where
i=1,2 (treatments, placebo or three-way combination)
j=1,2 (sex, M or F)
k=1,2, . . . 10 (block)
Yᵢⱼₖ=count of parasite from animal in block k, treatment i, and sex j
μ=overall mean
b=random effect of block k
βⱼ=effect of sex j
β(b)ⱼₖ=random effect of block k within sex j,
αᵢ=effect of treatment i
αβᵢⱼ=interaction of treatment i by sex j
αβ(b)ᵢⱼₖ=random effect of treatment i, block k, within sex j,
eᵢⱼₖ=random error term

Least squares geometric means of the transformed parasite counts were used to compare treatment to the negative control. The 0.05 level (p≦0.05) was used to compare main effect treatment means. Least squares geometric means for each treatment was back transformed and presented as mean counts.

Percent effectiveness for A. caninum was calculated across sex according to the following formula:

$$Percent Effectiveness = \frac{( Mean of Control - Mean of Treated )}{( Mean of Control )} \times 100$$

Mean of Control=Geometric mean of the number of parasites in the dogs from the appropriate control group.

Mean of Treated=Geometric mean of the number of parasites in the dogs from the appropriate treated group.

Efficacy

A. caninum worms were recovered from all control dogs at necropsy. Dogs receiving the soft chew(s) containing the active ingredients had significantly (P<0.0001) reduced A. caninum present at necropsy compared with control dogs. Efficacy of the soft chew containing fenbendazole, ivermectin, and praziquantel based on reductions of the number of worms recovered at necropsy was 98.4% (Table 3).

TABLE 3
Recovery of Hookworms at Necropsy

| Treatment Group | Ancylostoma caninum |
|---|---|
| Placebo | |
| Geometric Mean | 17.60 |
| Standard Error | 0.17 |
| Fenbendazole, Ivermectin, Praziquantel | |
| Geometric Mean | 0.28* |
| Standard Error | 0.13 |
| Efficacy^{†} | 98.4% |

*Significant reduction in worm count versus controls (P < 0.001).
^{†}Percent efficacy = [Geometric mean (control) − geometric mean (treated)/geometric mean (control)] × 100.

Adverse Events

There were no treatment related adverse events observed during the study. Pre- and post-treatment blood values were acceptable.

Protocol Deviations

All dogs had blood drawn on Study Day 5 instead of Study Day 7 as listed in the protocol because Study Day 7 fell on a Saturday. Drawing blood on Day 5 instead of Day 7 did not impact the study in any way.

The protocol indicates that a randomized complete block design was used, however the statistical methodology described is based on a split plot design. The statistical data that was generated followed a split plot design. The incorrect terminology did not impact the statistical findings of the study.

The protocol indicates that the dogs will be blocked according to fecal egg count within sex. However, when the randomization was generated the assumption was that the dogs were not blocked within sex. The treatment was still randomized and therefore this deviation did not impact the statistical findings of the study.

This study demonstrated that a single treatment of a palatable fenbendazole, ivermectin, and praziquantel combination soft chew product administered at a single dose of 100 mg fenbendazole, 6 mcg ivermectin and 5 mg praziquantel per kg body weight was efficacious against induced A. caninum infections.

### Example 2

Another was done to assess the efficacy of a palatable fenbendazole, ivermectin and praziquantel combination soft chew product administered at a single dose of 100 mg fenbendazole, 6 mcg ivermectin and 5 mg praziquantel per kg body weight against natural infections of Trichuris vulpis. Twenty dogs (10 males and 10 females) were confirmed as having T. vulpis infections by three separate fecal egg counts by Study Day—2. The dogs were ranked in descending order, within sex, according to their mean fecal egg count and then randomly assigned to one of two groups: Group 1 received a single treatment of the soft chews containing the active veterinary product and Group 2 received a single treatment of the placebo control product. All of the dogs were administered treatment on Study Day 0. On Study Day 7, all dogs were euthanized and the intestinal contents were collected and the number of whipworms present was counted. The reduction in T. vulpis worm recoveries based on geometric means relative to the controls was 99.8%. No treatment-related adverse events were observed. This study demonstrated that a single treatment of a palatable fenbendazole, ivermectin and praziquantel combination soft chew product administered at a single dose of 100 mg fenbendazole, 6 mcg ivermectin and 5 mg praziquantel per kg body weight was efficacious against natural T. vuplis infections.

The objective of this exploratory study was to assess the efficacy of a palatable fenbendazole, ivermectin and praziquantel combination soft chew product administered at a single dose of 100 mg fenbendazole, 6 mcg ivermectin and 5 mg praziquantel per kg body weight against natural infections of T. vulpis.

Materials and Methods

The dogs were randomly allocated to one of two treatment groups in a split plot design. The whole plot was sex and the subplot was treatment, which was assigned to the dogs in a randomized complete block design. The dogs within each sex were ranked by the mean fecal egg count in descending order. Blocks contained two consecutively ranked dogs within each sex. The treatment was randomly assigned to dogs within each block where there was equal allocation of treatment to sex. Dogs received either a single treatment of the soft chews containing the active veterinary product or a single treatment of the placebo control product.

Treatments

Veterinary Product

The active ingredients were fenbendazole (100 mg/kg), ivermectin (6 mcg/kg) and praziquantel (5 mg/kg). The batch number of this experimental formulation was PC005.094. The complete formulation is on file with the Sponsor. The veterinary product was stored in a dry place at controlled room temperature and was protected from light.

Control Product

The control product did not contain any active ingredients. The batch number for the control product was PC005.091. The control product was stored in a dry place at controlled room.

Dosage and Administration

The investigational and control products were in a chewable solid dosage form and each soft chew weighed approximately 5.2 grams or 5.4 grams, respectively. The veterinary product contained the active ingredients in sufficient quantity to treat up to 24.5 pounds of body weight. The dogs' weights ranged from 27.2 to 72.7 pounds. Dogs received a single oral treatment of either the veterinary product or the control product. Food was removed from the dogs the night before dosing, and then food was offered approximately 30 minutes prior to dosing. Of those dogs assigned to the veterinary product, four dogs were each administered two chews and the remaining six dogs were each administered three chews.

Study Procedures

| Study Day | Activity |
|---|---|
| −14 | Dogs placed in individual kennels or cages and start |
| | of acclimation. |
| −3 | Physical exams. Blood samples for hematology |
| | and serum chemistry. |
| −5 to −3 | Three fecal samples collected and parasite egg |
| | counts performed. Mean FEC recorded. |
| −1 | Weigh dogs for study. Assignment of dogs to |
| | treatment group. |
| 0 | Administer treatment and begin hourly observations |
| | for 4 hours then daily observations for adverse |
| | events. |
| 1-7 | Daily observations |
| 5 | Blood samples collected. |
| 7 | Necropsies and parasite collections, parasite counts |
| | may be performed later. |

Randomization/Allocation to Treatment Group

Enumeration of T. vulpis eggs in fecal samples was done for each dog on three separate days by Study Day—2. Dogs were then ranked by their mean fecal egg counts within sex in descending order and randomly assigned to a treatment group (five males and five females per group).

Study Variables and Observations

Pre-treatment physical examinations and blood samples were collected by Study Day—7 to establish that dogs were healthy, except for parasitism, prior to inclusion in the study. Blood samples were also collected prior to euthanasia to compare hematology and serum chemistry parameters to the initial values. The blood samples were sent to a clinical pathology laboratory for a complete blood count and serum chemistry profile.

All dogs were observed once per hour for adverse events for 4 hours after administration of the veterinary and control product. After this the dogs were observed once daily until they were euthanized (Study Day 7).

As suggested in Guidance for Industry 111, Effectiveness of Anthelmintics: Specific Recommendations for Canine VICH GL19, the primary efficacy variable was the number of T. vulpis worms recovered at necropsy. Counts of T. vuplis were determined as described in the Appendix II. The counts were documented and are included in the study data. There were no secondary variables.

Analytical Methods

Standard laboratory equipment was used in the collection of samples and for the identification of parasites.

Statistical Methods

The experimental unit was the individual dog.

Parasite counts were transformed by taking the natural logarithm of the count of intestinal parasites recovered from each animal +1. One was added to the count to prevent an undefined result for those dogs with no parasites. The transformed counts were analyzed using the Proc Mixed procedure in the SAS® statistical software by modeling the counts with a mixed model. Treatment and sex were modeled as fixed effects and block was modeled as the random effect. The model is listed below:
Log(Yᵢⱼₖ+1)=μ+bₖ+αᵢ+βⱼ+αβᵢⱼ+αbᵢₖ+βbⱼₖ+eᵢⱼₖ
where
i=1,2 (treatments, placebo or three-way combination)
j=1,2 (sex, M or F)
k=1,2, . . . 10 (block)
Yᵢⱼₖ=count of parasite from animal in block k, treatment i, and sex j
μ=overall mean
b=random effect of block k
βⱼ=effect of sex j
β(b)ⱼₖ=random effect of block k within sex j,
αᵢ=effect of treatment i
αβᵢⱼ=interaction of treatment i by sex j
αβ(b)ᵢⱼₖ=random effect of treatment i, block k, within sex j,
eᵢⱼₖ=random error term

Least squares geometric means of the transformed parasite counts were used to compare treatment to the negative control. The 0.05 level (p≦0.05) was used to compare main effect treatment means. Least squares geometric means for each treatment was back transformed and presented as mean counts.

Percent effectiveness for T. vulpis was calculated across sex according to the following formula:

$$Percent Effectiveness = \frac{( Mean of Control - Mean of Treated )}{( Mean of Control )} \times 100$$

Mean of Control=Geometric mean of the number of parasites in the dogs from the appropriate control group.

Mean of Treated=Geometric mean of the number of parasites in the dogs from the appropriate treated group.

Results

Efficacy

T. vulpis worms were recovered from all control dogs at necropsy. Dogs receiving the soft chew(s) containing the active ingredients had significantly (P<0.0001) reduced T. vulpis present at necropsy compared with control dogs. Efficacy of the soft chew containing fenbendazole, ivermectin, and praziquantel based on reductions of the number of worms recovered at necropsy was 99.8% (Table 3).

TABLE 3
Recovery of Trichuris vulpis at Necropsy

| Treatment Group | Trichuris vulpis |
|---|---|
| Placebo | |
| Geometric Mean | 184.86 |
| Standard Error | 0.278 |
| Fenbendazole, Ivermectin, Praziquantel | |
| Geometric Mean | 0.31* |
| Standard Error | 0.278 |
| Efficacy^{†} | 99.8% |

*Significant reduction in worm count versus controls (P < 0.001).
^{†}Percent efficacy = [Geometric mean (control) − geometric mean (treated)/geometric mean (control)] × 100.

Adverse Events

There were no treatment related adverse events observed during the study.

Pre- and post-treatment blood values were acceptable.

Protocol Deviations

The protocol indicates that a randomized complete block design was used, however the statistical methodology described is based on a split plot design. The statistical data that was generated followed a split plot design. The incorrect terminology did not impact the statistical findings of the study.

The second paragraph in Appendix B (Procedure for Intestinal Helminth Collection) of the protocol was not applicable to the cecum/colon examination. The paragraph came from a site Standard Operating Procedure and should not have been included in the Appendix. This deviation did not affect the outcome of the study.

### CONCLUSIONS

This study demonstrated that a single treatment of a palatable fenbendazole, ivermectin, and praziquantel combination soft chew product administered at a single dose of 100 mg fenbendazole, 6 mcg ivermectin and 5 mg praziquantel per kg body weight was efficacious against natural T. vulpis infections.

While the invention has been described in connection with specific embodiments and examples thereof, it will be understood that it is capable of further modifications and the appended claims are intended to cover any variations, uses, or adaptations of the invention following, in general, the principles of the invention and including such departures from the present disclosure as come within known or customary practice within the art to which the invention pertains and as may be applied to the essential features hereinbefore set forth whether now existing or after arising. Further, while embodiments of the invention have been described with specific dimensional characteristics and/or measurements and or components, it will be understood that the embodiments are capable of different dimensional characteristics and/or measurements and/or components without departing from the principles of the invention and the appended claims are intended to cover such differences. Furthermore, all patents, printed publications, and the like mentioned herein are herby incorporated by reference. In jurisdictions not allowing incorporation by reference, Applicants will add the matter to the specification.

1. A composition for the treatment of parasites comprising an effective amount of the components comprising a benzimidazole, an avermectin and praziquantel.

2. The composition of claim 1 wherein the active ingredients are delivered in a soft chew comprising a flavoring component of between about 0.1 to about 50 percent, a starch component of between about 5.0 to about 60 percent, a sugar component of between about 5.0 to about 75 percent, an oil component of between about 1.0 to about 40 percent, and a first additive wherein the moisture content is less than about 15 percent and wherein the soft chew is formed by knockout, the soft chew is not an extrudate.

3. The composition of claim 2 further comprising at least one additive selected from the group consisting of a pharmaceutical, a nutraceutical, a vitamin, a mineral, and a filler.

4. The composition of claim 2 wherein the flavoring component is selected from the group consisting of fruit, meat, vegetable, cheese, cheese-bacon and/or artificial flavorings.

5. A pharmaceutical dosage form comprising an effective amount of Fenbendazole, Ivermectin, and Praziquantel for the substantial elimination of Toxocara canis, Ancylostoma caninum, Trichuris vulpis, Dipylidium caninum and Dirofilaria immitis from an organism.

6. A method for treating an organism against Toxocara canis, Ancylostoma caninum, Trichuris vulpis, Dipylidium caninum and Dirofilaria immitis comprising administering an effective amount of a composition of claim 1.

7. A process for introducing at least one additive to an organism comprising offering the soft chew of claim 1 to the organism, whereby, upon consumption, the additive is ingested by the organism.

8. The process of claim 7 wherein the organism is selected from the group consisting of a horse, cow, pig, goat, sheep, llama, deer, duck, chicken, dog, cat, lion, tiger, bear, an ox, buffalo, fish, human and the like.

9. A process for making a composition according to claim 2 comprising the steps of:
a. mixing a flavoring component, a starch component, a sugar component, and an oil component into a dough;
b. mixing a benzimidazole, an avermectin and praziquantel into the dough;
c. heating the dough;
forming the soft chew with a knockout, such that the soft chew is not an extrudate.

10. The process of claim 9 wherein the step of heating the components comprises heating the oil component prior to mixing.

11. The process of claim 9 wherein the step of forming the soft chew comprises moving the dough from a hopper to a press.

12. A process for delivering an additive to an organism comprising administering a soft chew according to claims 1 or 5 to an organism.

13. A process of forming a soft chew comprising the steps of:
a. mixing a flavoring component, a starch component, a sugar component, an oil component, and an additive into a dough;
b. heating the dough; and,
c. knocking out a soft chew.

14. The process of claim 13 wherein the step of knocking out is performed on a patty pressing machine.

15. The process of claim 13 wherein the soft chew further comprises additional additives.
